(19) United States
(12) Patent Application Publication
(10) Pub. No.: US 2003/0191427 A1
(43) Pub. Date: Oct. 9, 2003

(54) Breast band for hands-free breast pumping

(76) Inventors: Lisa A. Jay, Arlington, VA (US); Margaret A. LaShoto, Waltham, MA (US); Louis J. Foreman, Huntersville, NC (US)

Correspondence Address: JINAN GLASGOW, P O BOX 28539, RALEIGH, NC, 276118539

(21) Appl. No.: 10/116,936

(22) Filed: Apr. 5, 2002

(51) Int. Cl. A61M 1/06; A61M 37/00

(52) U.S. Cl. 604/24; 604/74

(57) ABSTRACT

A strapless hands-free breast band device of the present invention includes a unitary breast band having self-closing openings for receiving milk intake receptacles that is custom-fittable to the user. The self-closing openings are horizontal slit openings in an anisotropic breast band material that provide for adequate support of the milk intake receptacles without deflection, bending, or buckling of the band due to the weight of the receptacles when filling or filled with milk.

### BACKGROUND OF THE INVENTION

(1) Field of the Invention

The present invention relates generally to devices that facilitate breast pumping and, more particularly, to a breast band for hands-free breast pumping.

(2) Description of the Prior Art

A few prior art patents address hands-free breast pumping.

U.S. Pat. No. 5,616,125 to Cassandra N. Jelks describes an apparatus for simultaneously pumping milk from the right and left breast of a nursing mother comprising a nipple shield formed in a generally conical configuration with an exterior surface and an interior surface positionable over the nipple of a breast of a nursing mother. The shield has a centrally located aperture with a cylindrical coupling extension projecting from the exterior surface of the shield around the aperture. Further included is a pump. The pump has an input and an output. A flexible tube is provided and has an input end coupled to the conical extension of the shield and an output end coupled to the input of the pump. A housing is formed of a container with a separable cover. The container receives the pump and the end of the tube adjacent to its output end. A baby bottle is included and has an open top and positioned within the housing adjacent to the pair of pumps. A lid is removably positioned on the open top of the baby bottle with a large hole and a small hole for air exhaust. A supplemental tube couples the output of the pump to the large hole. A switch within the container is for activating the pump. A power source within the container with lines supplies power to the pump through the switch. Straps coupled to the container are positionable about the shoulders of the user to releasably secure the container to the back of the user.

U.S. Pat. No. 5,575,768 to Lockridge et al. describes a device for supporting a breast shield of a breast pump upon a woman's breast in a “hands-free” manner. This breast shield support includes a garment that is adapted to a woman's torso, and a mounting element formed with or attachable to the back of the breast shield. Elastic bands tied to loops on the garment are releasably affixed to the mounting element to support the breast shield on the exposed breast. A list for retrofitting the device to a breast pump assembly is advantageously provided.

U.S. Pat. No. 5,514,166 to Silver et al. describes a device and method for supporting a breast shield of a breast pump upon a woman's breast in a “hands-free” manner. Such a breast shield support includes, for example, a base member, such as a garment, that is adapted to be carried on a woman's torso. Part of the breast pump, such as the breast shield, is releasably attached to the base member and supports the breast shield on the exposed breast. Embodiments include corresponding fasteners on the breast shield and a brassiere, a harness-type strap arrangement for holding the shield against the breast and a breast shield which slips through a flap formed in a brassiere cup and is supported by the cup material.

U.S. Pat. No. 6,227,936 to Amelia Mendoza describes a hands-free pumping and nursing brassiere that operates similar to a regular nursing bra, yet differs in that it has a detachable hands free pumping panel positioned behind the nursing cup. To nurse, a woman detaches the nursing cup with the pumping panel attached thereto. To perform hand free pumping, the nursing cup is detached from the pumping panel, a breast shield is inserted from the back of the pumping panel through an opening in the pumping panel, so that it can be attached to the pump. The pumping panel is then reattached to the brassiere allowing the woman to pump her breast milk in a hands free manner.

U.S. Pat. No. 6,213,840 to Bonnifant Heeja Han describes a hands-free breast pump support bra and system to allow simple and effective breast milk expression from a lactating woman's breasts without the need of additional support, such as straps, bands, posts and mounting elements to support the weight of breast pump apparatuses. The support bra independently and as part of the system is made of a material of sufficient elasticity and strength that when worn firmly and frictionally holds against each breast the funnel-like portion of a breast pump during the complete breast milk expression operation. Slits are strategically located in both nipple areas of the bra's cups through which the funnel-like portions of breast pumps engage the breasts. Because of the elastic property of the bra's fabric, the fabric around the slits securely and simultaneously hold against each breast in a hands-free manner a funnel portion with a conventional breast milk bottle filled with milk and attached thereto.

U.S. Pat. No. 6,004,186 to Gretchen M. Penny describes an apparatus for securing the milk intake components of a suction device to the nipples of a nursing mother's breasts configured as a bandeau or tube top. Alternatively, the apparatus may be configured as a halter top. The apparatus includes a chest piece for positioning around a nursing mother's chest and two openings formed in the front of the chest piece for receiving the milk intake components of the suction device. Optional closures such as Velcro.®. strips, snaps, hooks and eyes or buttons/button holes located on the chest piece permit adjusting the fit of the apparatus around the mother's chest. The halter top configuration may include a neck strap for adjusting the fit around the nursing mother's neck. Optional closures located on the neck strap permit adjusting the fit of the neck strap around the nursing mother's neck.

The devices described above facilitate breast pumping are generally bra-like in nature, including arm straps and openings to allow for insertion of the breast pumps on the nipples. The apparatus taught by the '186 patent has at least two disadvantages. It teaches that the chest piece may be manufactured of a stretchable non-woven material or a non-stretchable, woven material. It, however, fails to teach an anisotropic chest piece, one that stretches in one direction yet not the other. Such anisotropy in a chest piece would provide stretching ability in one direction and stiffness in the opposite direction. In fact the '186 patent teaches away from anisotropy as it suggests that if the chest piece is made of stretchable fabric then a strip of supporting fabric should be permanently secured to the bottom and/or top edges using appropriate attachment means.

A second disadvantage of the '186 patent is that it teaches two permanent openings formed in the chest piece positioned over the locations of the underlying nipples of the mother's breasts. Such openings may cause uncomfort if worn by a woman on a regular basis as the sides of the opening may rub against the nipple causing nipple abrasions or swellings. The nipple may also move in an out of the openings causing the woman discomfort in terms of not only the possibility of rubbing against the material of the chest piece but also the changing temperatures. A permanent opening implies an asymmetry in temperature as all the breast except the nipple areas will be covered. Thus there exists a need for openings that may open and close. Moreover if the openings in the '186 patent are in stretchable material then they may require reinforcement or “finishing”. Such finishing would not be required where the openings are substantially horizontal and are formed in anisotropic material. In this case, the non-stretchability in a vertical direction would provide the necessary support for the opening when a breast pump is inserted. Thus, no reinforcement is necessary.

Thus, there remains a need for a hands-free breast apparatus that is anisotropic in nature. Additionally, there remains a need for closeability of the openings positioned at the nipple areas. Finally, there remains a need for openings that support the weight of a breast pump without the necessity of added reinforcements.

### SUMMARY OF THE INVENTION

The present invention is directed to a unitary, strapless hands-free breast band substantially rectangular in shape having a first direction, a length, and a second direction, and a height.

In the preferred embodiment, the breast band is composed of anisotropic material that stretches in the first direction but not in the second direction.

Preferably, the breast band includes an aperture or opening that is self-closing upon removal of the pump intake receptacle.

Still another aspect of the present invention is to provide a breast band that has substantially horizontal openings positioned at the nipple areas. Accordingly, one aspect of the present invention provides for closeable openings.

In a more preferred embodiment the breast band is compression molded such as to fit the contours of women's breasts.

These and other aspects of the present invention will become apparent to those skilled in the art after a reading of the following description of the preferred embodiment when considered with the drawings.

### BRIEF DESCRIPTION OF THE DRAWINGS

FIG. 1 is a full-frontal view of the preferred embodiment of the hands-free breast band.

### DETAILED DESCRIPTION OF THE PREFERRED EMBODIMENTS

In the following description, like reference characters designate like or corresponding parts throughout the several views. Also in the following description, it is to be understood that such terms as “forward,” “rearward,” “front,” “back,” “right,” “left,” “upwardly,” “downwardly,” and the like are words of convenience and are not to be construed as limiting terms.

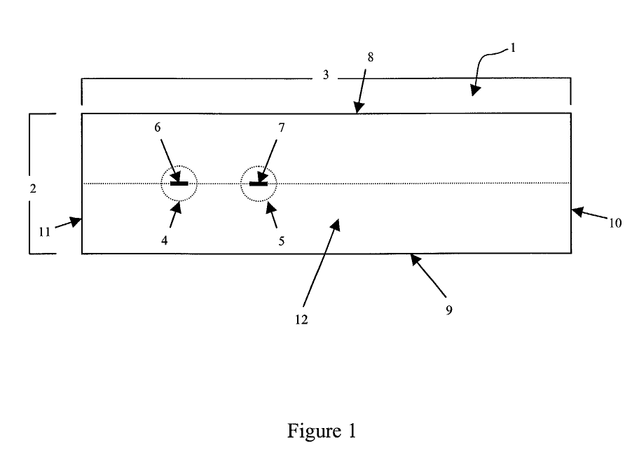

Referring now to the drawings in general, the illustrations are for the purpose of describing a preferred embodiment of the invention and are not intended to limit the invention thereto. The device of the present invention is configured as a unitary breast band having self-closing openings for receiving milk intake receptacles. FIG. 1 shows the preferred embodiment, a strapless hands-free breast band 1 substantially rectangular in shape having a top edge 8 and bottom edge 9 both of length 3 and a first end 11 and a second end 10 both of height 2. The breast band 12 is the primary component of the apparatus. In one embodiment according to the present invention, the breast band 12 may be manufactured as a single strip of fabric having an essentially constant width along the entire band 12 (see FIG. 12). In an alternative embodiment, the breast band may be tapered at the back region to provide increased comfort and freedom of movement. Ideally, the band 12 is approximately thirty inches (30″) long and approximately eight inches (8″) to ten inches (10″) wide. Alternatively, the band 12 may be manufactured as a single strip of fabric.

Also shown in FIG. 1 on the breast band 12, are breast cup regions 6, 7 which may be contoured or capable of being contoured to accommodate different breast cup sizes. Notably, while the initial material may be planar, the cups may be individually shaped or contoured or molded about the woman's breasts at the first wearing of the band in order to provide for more customizeable shaping of the band for the user.

In a preferred embodiment, the breast band is composed of anisotropic material that stretches in the first direction, length, but not in the second direction, height. The chest piece 12 may be manufactured of a braided or woven anisotropic material. The anistropic material may be of various thicknesses. The anistropic material includes fabric, foam or a combination thereof. In a preferred embodiment the fabric is breathable. Breathable means the material allows for ample ventilation. Air may pass through the material to the skin of the wearer. As such, a preferred embodiment of the invention includes a mesh-like fabric, for example, COOLMAX fabric, which is commercially available by the DuPont company. The material may be a 3-D engineered material, formed by 3-D fiber engineering methods providing for true 3D weaving involving three orthogonal, non-interlacing yarn systems, as formed by the methods set forth in U.S. Pat. No. 5,085,252 issued Feb. 4, 1992 to Mohamed, et al. for METHOD OF FORMING VARIABLE CROSS-SECTIONAL SHAPED THREE-DIMENSIONAL FABRICS which is incorporated herein by reference in its entirety. Also, U.S. Pat. No. 5,465,760 issued Nov. 14, 1995 to Mohamed, et al. for MULTILAYER THREE-DIMENSIONAL FABRIC AND METHOD FOR PRODUCING is also incorporated herein by reference in its entirety. Alternatively, the material may include layered material, in particular laminated layers that form the anisotropic material for the band according to the present invention. By way of example, other materials that may be appropriately used for the breast band of the present invention include neoprene, polypropylene, breatheable stretch spacer material, and the like. Furthermore, an anti-microbial agent may be added to the material or fibers that are used in forming the material that forms the band body; this added anti-microbial agent advantageously prevents bacteria from developing on the band during use and between cleanings, thereby making the product safer, in particular for extended use.

In another preferred embodiment the breast band is continuous. During use of the apparatus, the breast band 12 is wrapped around the nursing mother's chest. The breast band 12 may be constructed as a single strip of material having a first end 11 and a second end 10 (FIG. 1). The first end 11 and second end 10 of the breast band may be permanently secured to each other, thereby creating a single, tubular-shaped breast band 12 (FIG. 1). Any means known in the art to permanently connect multiple pieces of fabric, such as a needle and thread, a heat-activated fusible material, or a fabric adhesive such as glue may be used to permanently secure the first end 11 and second end 10 of the breast band 12 together to create the tubular shape of the breast band 12. The secured ends of the breast band 12 create a seam in the breast band 12. In a preferred embodiment of the present invention, the releasably fastening seam is oriented substantially vertically on the nursing mother's side. The seam may also be located in other positions on the breast band 12, such as in the front between the breasts. In another case, the seam may be angled slightly off the vertical such that the first end 11 and the second end 10 of the breast band 12 would also be angled or slanted in an appropriate direction. If the material used to construct the apparatus is tubular in nature at the start of construction the seam would be unnecessary and therefore absent. Alternatively, the breast band may be incorporated into a garment, such that the band is still oriented around the woman's chest region, but it is sewn or otherwise incorporated into a garment that is pulled on overhead, like a t-shirt. In such case, there would additionally be slits in the garment that correspond to the slits in the breast band.

Alternatively, the breast band 12 may be wrapped around the nursing mother's chest as a single strip of fabric having two ends that are temporarily secured to each other. The first end 11 and the second end 12 of the breast band 12 may be temporarily secured to each other using connection or fastener means known in the art. Suitable fastener means include hook and loop fasteners, e.g., VELCRO closures, buttons and button holes, hook and eye closures, snaps or fabric extensions that can be tied. If temporary securing of the breast band 12 is desired, one component of the fastener means, e.g., hook is secured to the first end 11 of the breast band 12 and the complementary component of the fastener means, e.g., eye is secured to the second end 10 of the breast band 12.

When the unitary breast band 12 is wrapped around the nursing mother's chest or upper torso region, the components of the releasable closure are engaged or mated to temporarily fasten the first end 11 and the second end 10 of the breast band 12 together. When the mother has concluded the milk extraction procedure, the components of the closure means are disengaged, freeing the first end 11 and the second end 10 of the breast band 12. In conjunction with subsequent milk extraction procedures, the steps described above are repeated.

With the embodiments of the present invention having permanent or temporary fastening of the first end 11 and second end 10 of the breast band 12, the breast band 12 must fit snugly and continuously around the nursing mother's chest. A secure, tight fit of the breast band 12 around the mother's chest holds the milk intake component of the suction devices snugly against the nipples of the mother's breasts and prevents leakage around the milk intake component of the suction devices. The breast band 12 does not provide support for the breasts. Instead, breast band 12 provides support for the suction devices and connected milk intake receptacles.

In another preferred embodiment the breast band is form-fitting. Form-fitting means the band is constructed and arranged to fit snugly around the mother's chest and breasts and accommodate changes in breast size without providing support for the nursing mother's breasts. Form-fitted bands form around the contours of the wearer of the breast band. A nursing mother often notices significant changes (increases and/or decreases) in her chest or brassiere cup size between the first month of her pregnancy and the last month of breast-feeding. The breast band 12 is not required to be constructed to fit a particular brassiere chest or cup size. However, the breast band 12 can be constructed as form-fitting in that it adjusts to snugly fit the nursing mother during the increases and decreases in her chest or brassiere cup size during pregnancy and breast-feeding. Ideally, the breast band 12 is constructed in generally accepted industry sizes (e.g., small, medium and large) to accommodate nursing mothers having various chest and cup sizes. In addition, the breast band 12 may be manufactured to custom fit a particular torso size, if desired.

In more preferred embodiment the breast band is compression molded such as to fit the contours of women's breasts. Compression molding means heat and pressure has been applied to the material of the band when placed on a mold in the form of a woman's breast; alternatively, the band material be formable or moldable on the user's breasts at the first use or wearing, wherein body heat may provide the material with some memory or substantial shape-retention due to temperature-sensitive fibers and/or material treatment, such as a coating or other chemical treatment application to provide at least some memory qualities to the material. Where molds are used to pre-form the band to include contoured or contourable regions 6,7, the molds may be preformed for different breast sizes. Thus compression molded bands may represent different breast sizes.

Preferably, the breast band includes an aperture or opening that is self-closing upon removal of the pump intake receptacle. In a preferred embodiment the breast band has substantially horizontal openings or apertures 4 and 5 positioned approximately at each of the nipple areas. Two openings or apertures 4 and 5 are formed in the breast band 12 as slits that are cut into the fabric; importantly, these slits are self-closing upon removal of the pump intake receptacles at the completion of milk expression. The self-closing characteristic is only possible where the openings are slits and the fabric is anisotropic, with stretch properties in the longitudinal direction around the band to provide for a snug fit of the band on the woman's chest area and the perpendicular or height direction is non-stretchy or at least partially stiff to prevent buckling, bending, or overlapping of the band vertically. It is the anisotropic characteristics of the material that provide for the self-closure of the openings 4,5 when the receptacles are removed. No overlap of the band material or covering layer of fabric over the openings is required for the self-closure to permit coverage of the nipple area when the woman is not expressing milk with a pumping device into milk intake receptacles. Significantly, the openings 4 and 5 are coaxial, i.e., they are positioned along a common axis that runs longitudinally along the breast band length direction, and the openings are spaced apart in order to be properly positioned in the breast band 12 over the locations of the underlying nipples of the mother's breasts. Ideally and importantly, the openings are substantially horizontal; this substantial horizontality of the openings is significant and critical for supporting the milk-intake devices, particularly as they become filled with milk. As the openings are present horizontally in the anisotropic material, the stiffness in the second direction (h) used to support the milk-intake devices is not undermined. The stiffness prevents the milk-intake devices from falling forward when the openings are substantially horizontal. If the openings were in the vertical direction the stiffness of the anisotropic material used to support the milk-intake devices would be undermined. In such a case as substantially vertical openings, intake devices would be likely to fall forward and disengage the breast band. These substantially horizontal openings in the anisotropic material can support between about 0 to 16 ounces without deflection, bending, or buckling and without losing the placement of the receptacles with respect to the breasts and the nipples in particular, which could cause difficulty in pumping due to temporary loss of suction. Thus, the horizontal slit openings in the band provide for self-closure when receptacles are not inserted through them for milk expression and the horizontal slit openings in the anisotropic breast band material provide for adequate support of the milk intake receptacles without deflection, bending, or buckling of the band due to the weight of the receptacles when filling or filled with milk.

Each opening 4 and 5 must be able to fit snugly around the milk intake component of the suction device, but also stretch enough for the milk intake component to fit inside the opening. Slit openings 4 and 5 are approximately between about 1½ and about four (4) inches in length is suitable and ensures stability; more preferably the openings are between about 1½ inches and about three (3) inches.

In another preferred embodiment the breast band has closeable openings that are sealable. While the invention already provides for self-closing openings as set forth in the foregoing, it may be additionally advantageous to provide sealable, releaseable and resealable openings for extended wear of the band. By way of example but not limitation, one way of releasably sealing the openings includes zippered sealing. Often women may want to wear the breast band for convenience rather than taking it on and off every time a baby needs to be fed. As such, permanently open openings as in the prior art may cause discomfort if worn by a woman on a regular basis since the sides of the opening may rub against the nipple causing nipple abrasions or swellings. The nipple may also move in an out of the openings in prior art devices, causing the woman discomfort in terms of not only the possibility of rubbing against the material of the breast band but also the changing temperatures. A permanently opened opening as in the prior art also causes an asymmetry in temperature as the entire breast except the nipple areas is covered by the band. A closing means such as the self-closing slit openings of the present invention and further a releaseably sealable closing of the openings such as a padded or lined zipper avoids this problem.

Although preferred and alternative embodiments of the present invention have been illustrated in the accompanying Drawings and described in the foregoing Detailed Description of the Invention, it will be understood by those skilled in the art that the invention is not limited to the embodiments disclosed, but is capable of numerous modifications, rearrangements and substitutions of the component parts and elements without departing from the spirit of the invention.

Certain modifications and improvements will occur to those skilled in the art upon a reading of the foregoing description. By way of example, other materials may be used for the breast band to provide specialized functionality, e.g., breatheability. Also, it is possible to utilize one of the apertures on the breast band for mechanized milk expression while either not engaging the other side or directly breast feeding on the other side. Additionally, the band could be manufactured in a one-size-fits-all version to optimize production costs; for this embodiment of the present invention, a uniformly sized band is provided with a multiplicity of VELCRO-type fasteners at predetermined locations on the band for providing releaseable securement for a variety of women's chest sizes. A measuring device would be used for the woman to custom-fit the band to her chest dimensions, including the location of the slits for best accommodating her breasts for milk expression using the device according to the present invention. A marker line or alignment indicator would be provided for the user making slits for the milk receptacles, as set forth in the foregoing, thereby providing for a custom fitted band. All modifications and improvements have been deleted herein for the sake of conciseness and readability but are properly within the scope of the following claims.

We claim:

1. A unitary, strapless breast band for securing two milk intake components or receptacles of a suction device to the nipples of a nursing mother's breasts, comprising:
a substantially rectangular anisotropic material for positioning around the nursing mother's chest, said substantially horizontal anisotropic material having a top edge and a bottom edge, and a first end and a second end;
two self-closeable coaxial openings formed in said substantially rectangular chest piece for entrance and non-entrance through and holding of the milk intake components of said suction device therethrough.

2. The band according to claim 1 wherein the material is form-fitting without the necessity of additional support.

3. The band according to claim 1 wherein the anisotropic material is compression molded.

4. The band according to claim 1 wherein the closeable openings are releasably sealable.

5. The band according to claim 1 wherein the material is selected from the group consisting of fabric, foam and combinations thereof.

6. The band according to claim 5 wherein the material is 3-D engineered material.

7. The band according to claim 5 wherein the material is breatheable.

8. The band according to claim 7, wherein the breatheable material is a breathable stretch spacer material.

9. The band according to claim 1 wherein the material is laminated fabric.

10. The band according to claim 1 wherein the first and second ends of the band are secured by a releasable closure.

11. The band according to claim 10 wherein the releasable closure is adjustable.

12. The band according to claim 10 wherein the releasable closure is selected from the group consisting of hook and loop, VELCRO, buttons and button holes, hook and eye closures, snaps and fabric extensions.

13. The band according to claim 10, wherein the releasable closure is located on a side of the band.

14. The band according to claim 1, wherein the material includes an anti-microbial agent.

15. The band according to claim 1, wherein the material includes a memory-retention component for providing a custom-shaping for the particular woman using the band.

16. The band according to claim 1, wherein the band further includes contourable regions for providing a band having a predetermined breast cup size.

17. A custom-fitted breast band for securing two milk intake components or receptacles of a suction device to the nipples of a nursing mother's breasts, comprising:
a formable material having memory for providing shape-retention of the nursing mother's breasts after the band is initially positioned around the nursing mother's chest, said material having a top edge and a bottom edge, and a first end and a second end;
two self-closeable coaxial openings formed in said substantially rectangular chest piece for entrance and non-entrance through and holding of the milk intake components of said suction device therethrough.

18. The band according to claim 17, wherein the band is releasably attachable to the woman's chest region.

19. The band according to claim 17, wherein the band is strapless.

20. The band according to claim 17, wherein the material is breatheable.
